(19) United States
(12) Patent Application Publication
(10) Pub. No.: US 2025/0012783 A1
(43) Pub. Date: Jan. 9, 2025

(54) HOMOGENEOUS IMMUNOASSAY METHOD

(71) Applicant: Belgian Volition SRL, Isnes (BE)

(72) Inventors: Rémi RABEUF, Isnes (BE); Mark Edward ECCLESTON, London (GB)

(21) Appl. No.: 18/705,460

(22) PCT Filed: Oct. 28, 2022
(86) PCT No.: PCT/EP2022/080215

§ 371 Date: Apr. 26, 2024

(30) Foreign Application Priority Data
Oct. 29, 2021 (EP) 21306520.4
Jan. 28, 2022 (EP) 22305100.4

(51) Int. Cl. G01N 33/53; G01N 33/574

(52) U.S. Cl. CPC G01N 33/5306; G01N 33/57407; G01N 2800/26

(57) ABSTRACT

The invention relates to detecting the concentration of chromatin fragments in a body fluid sample using a homogeneous immunoassay (HIA) method.

### FIELD OF THE INVENTION

The present invention relates to homogeneous immunoassay methods for measuring extracellular chromatin fragments, along with kits for performing such methods.

### BACKGROUND OF THE INVENTION

Acute organ failure due to infectious or sterile sepsis and septic shock are leading causes of death. Sepsis is a preventable, life-threatening condition marked by severe organ dysfunction. According to the Global Report on the Epidemiology and Burden of Sepsis issued by the World Health Organisation (ISBN: 978-92-4-001078-9), in 2017 it was estimated that sepsis had affected 49 million individuals and was related to approximately 11 million potentially avoidable deaths worldwide, accounting for approximately 20% of all-cause deaths globally. The largest contributors to sepsis incidence and mortality among all age groups were diarrhoeal diseases and lower respiratory infections, respectively. Nearly half of all sepsis-related deaths were complications of injuries and non-communicable diseases and 41% (20 million) of all global sepsis cases occurred among children under five years of age.

Nucleosomes are released into the circulation following fragmentation of chromatin upon cell death. Many infections initiate cell death through a variety of mechanisms (such as cell binding and entry, endosomal TLR3 activation and gene expression) thereby increasing the number of circulating nucleosomes in the blood (Danthi et al., Annu. Rev. Virol. (2016) 3:533-53). In particular, NETosis is a form of neutrophil cell death where neutrophils release neutrophil extracellular traps (NETs), which can capture and kill bacteria and other pathogens to prevent them from spreading. Recent studies have shown that extracellular nucleosomes and NETs have been associated with the progression of several diseases, including sepsis. Whilst the best described extracellular traps (ETs) are produced by neutrophils, other immune cells also produce ETs.

Current assays for NETs include myeloperoxidase (MPO), neutrophil elastase (NE), cell free DNA (cfDNA) and nucleosomes. However, none of these are used clinically. The reasons for this include that patients with systemic inflammatory response syndrome or sepsis may develop multi-organ failure and deteriorate rapidly or unexpectedly and decisions regarding their clinical treatment must be made in a timely manner. The current measurement methods available are manual immunoassay kits that require several hours to perform. In addition these assays are not performed in real time but performed in assay batches up to several days after blood sample collection. So, while NETs levels have been shown to have the potential for use as a biomarker in sepsis, there are currently no assay methods for nucleosomes or other chromatin fragments reported for low cost near patient methods that generate results with a rapid turnaround time and for use to make clinical decisions in real time.

Holdenrieder et al., Int. J. Cancer (2001) 95:114-120 previously described detecting the level of nucleosomes in serum samples of patients with benign and malignant diseases. The epigenetic composition of circulating cell free nucleosomes in terms of their histone modification, histone variant, DNA modification and adduct content have also been investigated as blood based biomarkers in cancer, see WO 2005/019826, WO 2013/030577, WO 2013/030579 and WO 2013/084002. Circulating cell free nucleosome levels are particularly highly elevated in haematological cancers, both in humans (WO2021110776) and in other animals (Dolan et al. BMC Veterinary Research (2021) 17:276 and Wilson-Robles et al. BMC Veterinary Research (2021) 17:231).

There remains a need in the art to provide a rapid turnaround time test needed to diagnose and monitor patients and to guide treatment in real time.

### SUMMARY OF THE INVENTION

According to a first aspect, there is provided a method of detecting the concentration of extracellular traps (ETs), chromatin fragments and/or nucleosomes in a body fluid sample comprising analysing the body fluid sample using a homogeneous immunoassay (HIA) method and using the results obtained from the analysis to determine the concentration of ETs, chromatin fragments and/or nucleosomes in the body fluid sample.

According to a further aspect of the invention, there is provided a method of monitoring the progress of a disease in a subject, comprising:
(i) detecting the concentration of ETs, chromatin fragments and/or nucleosomes in a body fluid sample obtained from the subject using the method defined herein;
(ii) repeating step (i) on one or more occasions; and
(iii) using any changes in the concentration of ETs, chromatin fragments and/or nucleosomes to monitor the progression of the disease in the subject.

According to a further aspect of the invention, there is provided a method of assigning a risk of an adverse outcome to a subject suffering from an infection, comprising:
(i) detecting the concentration of ETs, chromatin fragments and/or nucleosomes in a body fluid sample obtained from the subject using the method defined herein; and
(ii) using the concentration of ETs, chromatin fragments and/or nucleosomes detected to assign the likelihood of an adverse outcome to said subject,
wherein a subject identified with a high likelihood of an adverse outcome is assigned for medical intervention.

According to a further aspect of the invention, there is provided a method of detecting a subject in need of medical treatment for sepsis or septic shock, comprising:
(i) detecting the concentration of ETs, chromatin fragments and/or nucleosomes in a body fluid sample obtained from the subject using the method defined herein; and
(ii) using the concentration of ETs, chromatin fragments and/or nucleosomes as an indicator that the subject is in need of medical treatment for sepsis or septic shock.

According to a further aspect of the invention, there is provided a kit for measuring the concentration of ETs, chromatin fragments and/or nucleosomes in a body fluid sample comprising: a reagent solution comprising one or more antibodies capable of specific binding to ETs, chromatin fragments and/or nucleosomes and a container suitable for use in a means to measure the degree of agglutination or precipitation caused upon antibody binding to determine the concentration of ETs, chromatin fragments and/or nucleosomes, and optionally including one or more buffer solutions.

According to a further aspect of the invention, there is provided a use of the kit as defined herein for identifying a subject being susceptible to sepsis or septic shock, or for monitoring a subject with sepsis or septic shock.

According to a further aspect of the invention, there is provided a use of the kit as defined herein for identifying a subject with a cancer, particularly a haematological cancer, or for monitoring a subject with a cancer, particularly a haematological cancer.

### BRIEF DESCRIPTION OF THE FIGURE

FIG. 1. A HIA nucleosome standard curve produced using the method described in EXAMPLE 1.

### DETAILED DESCRIPTION

There is a need for effective, rapid tests to diagnose and monitor acute conditions, such as sepsis. Current methods in the art generally measure blood-based biomarkers using sandwich immunoassay methods that are highly analytically specific (they can detect a single analyte among a mixture of thousands) and extremely analytically sensitive (they can detect and quantitate proteins present in minute amounts). However, only manual immunoassay methods are available commercially which require several hours to perform. The market leader is Roche who provide the Roche Cell Death ELISA which takes up to 2 working days to complete and the Roche Cell Death ELISAPLUS which takes a few hours. Moreover, assays must be performed in batches and one assay kit is sufficient for approximately 40 samples. Samples are therefore not analysed in real time but collected, frozen and stored until such time as sufficient samples have been accumulated for an analysis batch. Manual immunoassay methods are expensive, cannot be performed rapidly in real time and cannot be situated near to patients.

Most immunoassays are heterogeneous and require a separation step. For example, the separation may involve the separation of antigen-bound antibody from free antibody or the separation of antibody-bound antigen from free antigen. These assays are typically highly sensitive but slow as described above.

Analytical sensitivity was thought to be important for assays used for the measurement of chromatin fragments in the circulation, because normal human and animal levels are low. Generally, most normal human levels are low with a median of around 30 ng/ml with levels below 10 ng/ml for some subjects. Thus, simple assays such as homogeneous immunoassays (HIA) were thought to be unable to measure clinical chromatin fragment levels accurately and reproducibly in healthy subjects or subjects with mildly elevated nucleosome levels.

### Systemic Inflammatory Response Syndrome, Sepsis and Multi-Organ Failure

Recent studies have shown that there is a significant elevation in NET and chromatin fragment levels found in patients with acute conditions such as systemic inflammatory response syndrome (SIRS) or sepsis. The results indicate that an assay sensitivity of 250 ng/ml for (NETs derived) nucleosomes is sufficient to usefully monitor patient response to treatment (Stanford S. Jul. 17, 2021; 326363; PB0152 ISTH Academy). Similarly, an assay sensitivity of 1 μg/ml is sufficient to usefully distinguish patients who require, or who are likely to develop a requirement for, organ support (or who have or may develop multi-organ failure) (Rea C. Jul. 17, 2021; 326469; PB0268 ISTH Academy). These levels are within the measurable range of HIA methods. The inventors propose using HIA techniques to provide low cost, rapid, near patient methods that can be used to make clinical decisions in real time.

Thus, according to a first aspect of the invention, there is provided a method of detecting the concentration of extracellular traps (ETs), particularly neutrophil extracellular traps (NETs), chromatin fragments and/or nucleosomes in a body fluid sample comprising analysing the body fluid sample using a homogeneous immunoassay method and using the results obtained from the analysis to determine the concentration of ETs, chromatin fragments and/or nucleosomes in the body fluid sample.

Described herein is an immunoassay method in which extracellular chromatin fragments in a biological sample and a solution of detection antibodies are subjected to an agglutination reaction and the resulting agglutinated mixture is analysed for a change in an optical property (for example its absorbance or in its scattered light by irradiation) in order to determine the content of chromatin fragments in the biological sample. In particular, the immunoassay is a homogeneous immunoassay whereby the resulting optical property is measured directly from the agglutination/precipitation mixture without a need to separate un-bound reagents. Therefore, the claimed HIA method does not involve separation of (i) antibody-bound NETs or chromatin fragments (i.e. the analyte), or (ii) unbound antibody from the tested body fluid sample.

References herein to a “homogeneous immunoassay” or “HIA” refer to an immunoassay in which an analyte is detected without the need for a step of separating bound and un-bound reagents. Thus the immunoassay is a simple method that can be carried out in one reaction mixture, i.e. the method may be performed using a single antibody incubation step. This simplicity allows for rapid assays requiring only a few minutes to complete and easy automation. The instrumentation used may be located in a central laboratory or in a near patient location. Heterogeneous immunoassays, which will not be discussed in detail, require the use of a separation step to separate bound from un-bound reagents for measurement.

In one embodiment, the HIA method comprises:
(a) mixing the body fluid sample with a solution of one or more antibodies, wherein said antibodies are capable of specific binding to ETs, chromatin fragments and/or nucleosomes and cause agglutination or precipitation upon binding; and
(b) measuring the degree of agglutination or precipitation in the mixture;
wherein the measurement in step (b) is used to determine the concentration of ETs, chromatin fragments and/or nucleosomes in the body fluid sample.

An example of a HIA is immunoturbidimetry. Immunoturbidimetry is a homogeneous, label-free immunoassay method, which measures the change in optical transmittance of the reaction mixture at specific wavelengths. An alternative assay method, immunonephelometry, measures the change in light scattering instead of transmittance. Both methods use the specific binding of an antibody to an analyte, wherein the binding results in an agglutination reaction which causes an increase in the scattering of incident light in the sample which may be measured as an increase in scattered light or as a decrease in transmitted light. The change in light scattering is then compared to that produced by standard/control samples with known analyte concentrations.

For example, when a patient specimen containing an analyte is combined with a solution of detection antibodies, a complex between the analyte and the antibody will form. When light is directed through this suspension, a portion of the light will be transmitted and focused onto an optic device (e.g. a photodiode via an optical lens system). The amount of transmitted light observed by the optic device is indirectly proportional to the analyte concentration in the patient specimen. Therefore, a specimen that contains a high concentration of the analyte would transmit less light than a specimen that contains a low concentration of the analyte.

Conventional HIA reagents consist of a reaction buffer and a solution of antibodies. Under optimised conditions, the antibodies form a three-dimensional lattice with analytes in the sample to be analysed. An alternative method uses particle enhancement of the agglutination reaction. This usually involves adsorbing the antibodies on the surface of a bead (such as a latex bead, nanoparticles or colloidal gold) to give a stronger signal. Therefore, in one embodiment the one or more antibodies are present in a suspension of antibody coated particles. The one or more antibodies may be conjugated to the surface of the particle, such as the latex bead or nanoparticle. The antibodies may be directly linked to the bead or particle or alternatively may be indirectly linked through use of an affinity linker or tag, e.g. a biotin, avidin, streptavidin or His (e.g. hexa-His) tag.

Methods of the invention generally used an optical property to measure the degree of agglutination or precipitation. Therefore, in one embodiment, the degree of agglutination or precipitation is measured by absorbance, transmittance, reflectance, light scatter, fluorescence, or scintillation of the mixture.

Antibodies suitable for use in the immunoassay methods are known in the art. Such antibodies are capable of specific binding to analyte, i.e. the NETs, chromatin fragments and/or nucleosomes. In one embodiment, the one or more antibodies are monoclonal antibodies. In an alternative embodiment, the one or more antibodies are polyclonal antibodies.

It will be clear to those skilled in the art that the terms “antibody”, “binder” or “ligand” as used herein are not limiting but are intended to include any binder capable of binding to particular molecules or entities and that any suitable binder can be used in the method of the invention.

The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody, a polyclonal antibody or a fragment thereof. It will be understood that if an antibody fragment is used then it retains the ability to bind the biomarker so that the biomarker may be detected (in accordance with the present invention).

In one embodiment, the methods described herein are used to measure the concentration of extracellular traps. In particular, the ETs may be neutrophil extracellular traps (NETs). NETs have been detected in a number of body fluid samples (see for example Saffarzadeh et al. (2012) PLOS ONE 7 (2): e32366) and any body fluid containing NETs may be tested for NETs or chromatin fragments using the methods described herein. Methods and uses described herein may be tested in body fluid samples, in particular blood, serum or plasma samples. Preferably, plasma samples are used. Plasma samples may be collected in collection tubes containing one or more anticoagulants such as ethylenediamine tetraacetic acid (EDTA), heparin, or sodium citrate, in particular EDTA.

In one embodiment, the methods described herein are used to measure the concentration of chromatin fragments. In one embodiment, the chromatin fragments measured by methods of the invention, comprise one or more nucleosomes. In one embodiment, the chromatin fragments and/or nucleosomes measured by methods of the invention, comprise an epigenetic feature of a cell free nucleosome. Therefore, the one or more antibodies may be capable of specific binding to the epigenetic feature of the cell free nucleosome.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361:25-62).

References to “nucleosome” may refer to “cell free nucleosome” when detected in body fluid samples. It will be appreciated that the term cell free nucleosome throughout this document is intended to include any cell free chromatin fragment that includes one or more nucleosomes. The cell free nucleosome may be mononucleosomes, oligonucleosomes, a constituent part of a larger chromatin fragment or a constituent part of a NET or a mixture thereof.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Methods and uses of the invention may measure the level of (cell free) nucleosomes per se. Therefore, in one embodiment the methods described herein are used to measure the concentration of nucleosomes, i.e. nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells. Nucleosomes also comprise DNA, therefore nucleosomes and ETs may be measured by methods of the invention using a binder of DNA, for example an anti-DNA antibody, either alone or in combination with binders of other epitopes found on ETs.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts.

Methods of the invention may detect the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone isoform, a modified nucleotide and/or proteins bound to a nucleosome in a nucleosome-protein adduct.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t. In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826.

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79:693). In one embodiment, the histone PTM is selected from citrullination or ribosylation. In a further embodiment, the histone PTM is H3 citrulline (H3cit) or H4 citrulline (H4cit). In a yet further embodiment, the histone PTM is H3cit.

A group or class of related histone post translational modifications (rather than a single modification) may also be detected. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti-ubiquitin antibodies. Antibodies to histone N-terminal domains, for example the N-terminal domains of H2A or H4 may be useful in the invention as these will confer a multi-dentate dimension to nucleosome assays.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

The chromatin exuded in NETs may contain, or be adducted to, or be decorated with, a number of proteins including, without limitation, myeloperoxidase (MPO), neutrophil elastase (NE), lactotransferrin, azurocidin, cathepsin G, leukocyte proteinase 3, lysozyme C, neutrophil defensin 1, neutrophil defensin 3, myeloid cell nuclear differentiation antigen, S100 calcium-binding protein A8, S100 calcium-binding protein A9, S100 calcium-binding protein A12, actin B, actin y, alpha-actin, plastin-2, cytokeratin-10, catalase, alpha-enolase and transketolase (Urban et al., PLOS Pathogens. (2009) 10: e1000639). Any nucleosome-protein adduct that occurs in NETs is a useful adduct for the detection of elevated levels of NETs in methods of the invention.

In one embodiment, the one or more antibodies are capable of specific binding to a protein associated with a neutrophil extracellular trap (NET). In a further embodiment, the protein associated with a NET is selected from myeloperoxidase (MPO) and neutrophil elastase (NE).

Polyclonal antibodies consist of multiple antibodies (produced by multiple clones) directed to bind different epitopes on the same target. The presence of multiple antibodies directed to bind multiple epitopes within the NETs is useful to maximise the degree of agglutination achieved. Therefore, in one embodiment of the invention one or more polyclonal antibodies, that may be capable of binding to multiple epitopes available on an ET or a nucleosome, is used as the binding agent.

Similarly, the use of multiple monoclonal antibodies, directed to multiple epitopes within the NETs (including, for example, different histone proteins, DNA, different histone modifications or other epigenetic features) may increase the degree of agglutination achieved. Therefore, in another embodiment of the invention, multiple (i.e. two or more) monoclonal antibodies are used as the binding agent. In this embodiment, the antibodies bind to different epitopes. In one embodiment, the HIA method comprises using two or more binding agents which bind to different epitopes, to cause agglutination or precipitation upon binding. In a further embodiment, the HIA method comprises: (a) mixing the body fluid sample with a solution of two or more binding agents (e.g. antibodies), wherein said two or more binding agents are capable of specific binding to different epitopes of ETs, chromatin fragments and/or nucleosomes and cause agglutination or precipitation upon binding; and (b) measuring the degree of agglutination or precipitation in the mixture; wherein the measurement in step (b) is used to determine the concentration of ETs, chromatin fragments and/or nucleosomes in the body fluid sample.

Therefore, multiple binding agents may be used which are directed to different epitopes within NETs. In one embodiment, the multiple binding agents (e.g. two or more binding agents, such as two or more antibodies) are capable of specific binding to different features of a cell free nucleosome. In a further embodiment, the different features may comprise one or more epigenetic features of a cell free nucleosome (e.g. a post-translational histone modification, a histone isoform, a modified nucleotide and/or proteins bound to a nucleosome in a nucleosome-protein adduct). For example, one of the binding agents may be directed to a histone protein epitope (e.g. histone H3 isoform H3.1) and the other binding agent is directed to a different feature (e.g. a nucleosome conformational epitope only present in intact nucleosomes containing histones and DNA).

### Diagnosis and Monitoring Methods

According to a further aspect of the invention, there is provided a method of monitoring the progress of a disease in a subject, comprising:
(i) detecting the concentration of ETs (such as NETs), chromatin fragments and/or nucleosomes in a body fluid sample obtained from the subject using the method as defined herein;
(ii) repeating step (i) on one or more occasions; and
(iii) using any changes in the concentration of ETs, chromatin fragments and/or nucleosomes to monitor the progression of the disease in the subject.

As described herein, methods of the invention can be used in situations requiring a fast result to allow clinical decision to be made in real time. Therefore, in one embodiment, the subject is in a critical care unit (also referred to as an intensive care unit).

The methods described herein also find particular use with patients suffering from systemic inflammatory response syndrome (SIRS). SIRS is an inappropriate response of the body involving an inappropriately high level of NETosis to an insult. The insult may be infectious or non-infectious including trauma, surgery, acute inflammation, ischemia, reperfusion, malignancy and many others. In one embodiment, the subject is suffering (or suspected to be suffering) from SIRS. In one embodiment, the subject is suffering (or suspected to be suffering) from an infection. In one embodiment, the subject is suffering (or suspected to be suffering) from sepsis or septic shock.

According to a further aspect of the invention, there is provided a method of assigning a risk of an adverse outcome to a subject suffering from an infection, comprising:
(i) detecting the concentration of ETs (such as NETs), chromatin fragments and/or nucleosomes in a body fluid sample obtained from the subject using the method as defined herein; and
(ii) using the concentration of ETs, chromatin fragments and/or nucleosomes detected to assign the likelihood of an adverse outcome to said subject,
wherein a subject identified with a high likelihood of an adverse outcome is assigned for medical intervention.

The methods of the invention may be directed to assigning a patient with a risk of an adverse outcome. Adverse outcomes include mortality and/or an acute event requiring immediate medical care, for example, hospitalisation (i.e. hospital treatment) and/or surgery. For many patients, infections are overcome by their own immune system without requiring medical intervention. However, in a number of patients, infections can proceed or increase in severity without being overcome by the immune system or the patient's own immune response to an infection may lead to an adverse outcome. Similarly, a patient with SIRS may be overcome by the condition potentially suffering multiple organ failure. For example, an adverse outcome may include an acute coronary or cardiac event (such as a myocardial infarction and/or stroke), acute multi- or single-organ failure (such as renal failure, liver failure and/or heart failure), onset of a debilitating acute condition and/or an acute respiratory condition (such as pneumonia, hypoventilation/bradypnea, acute respiratory distress syndrome (ARDS) severe acute respiratory syndrome (SARS), bronchiolitis and/or bronchitis). Thus, in one embodiment, the methods described herein assign a patient or subject with a risk of developing an acute respiratory condition. In a further embodiment, the acute respiratory condition is pneumonia. In a further embodiment, the acute respiratory condition is hypoventilation/bradypnea. In a yet further embodiment, the acute respiratory condition is acute respiratory distress syndrome (ARDS) and/or severe acute respiratory syndrome (SARS). The condition of a patient with sepsis is often expressed in terms of the organ failure assessment score (SOFA score). SOFA is used as a measure of organ function or organ failure to track the condition of a patient in critical care.

Assigning the patient with a risk of an adverse outcome may assign a near- or short-term risk or may assign a medium-term risk. A near- or short-term risk includes wherein the patient may develop an adverse outcome within 30 days, such as within 2 weeks or 14 days, within 1 week or 7 days or within 5 days or 1 day or hours of presentation of symptoms or of a positive diagnosis. Such near- or short-term risk may also include wherein the patient may develop an adverse outcome within 30 days, such as within 2 weeks or 14 days, within 1 week or 7 days or within 5 days or 1 day or hours of performing the methods described herein.

An example of an acute short term risk includes the development of NETs related complications to a COVID infection or sepsis requiring hospital treatment. The condition of a patient with COVID or sepsis may deteriorate rapidly over a few hours leading in severe acute cases to death within 12 hours. This is why patients with COVID, sepsis, SIRS, ARDS, SARS and similar conditions require a rapid, near patient monitoring method of the invention.

A medium-term risk includes wherein the patient may develop an adverse outcome more than 30 days after presentation of symptoms, a positive diagnosis and/or the performing of methods as described herein. An example of a medium term risk includes the development of so called long-COVID wherein the effects of a COVID infection may continue for many months.

Thus, in one embodiment, the methods described herein assign a patient or subject with a risk of developing an adverse outcome within 2 weeks, or 14 days, of presentation of symptoms or a positive diagnosis. In a further embodiment, the methods described herein assign a risk of developing an adverse outcome within 1 week, or 7 days, of presentation of symptoms or a positive diagnosis. In a yet further embodiment, the methods described herein assign a risk of developing an adverse outcome within 1 day or less of presentation of symptoms or a positive diagnosis.

If a subject is determined to not currently have a dangerously or inappropriately high level of NETs, then the invention may still be used for the purposes of monitoring disease progression for future development of a medical complication. For example, if the method comprises a sample from a subject determined to have a mild infection, then the NETs or chromatin fragment level measurements can be repeated at another time point to establish if the level has changed.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

Detecting and/or quantifying may be performed directly on the purified or enriched nucleosome sample, or indirectly on an extract therefrom, or on a dilution thereof. Quantifying the amount of the biomarker present in a sample may include determining the concentration of the biomarker present in the sample. Uses and methods of detecting, monitoring and of diagnosis according to the invention described herein are useful to confirm the existence of a disease, to monitor development of the disease by assessing onset and progression, or to assess amelioration or regression of the disease. Uses and methods of detecting, monitoring and of diagnosis are also useful in methods for assessment of clinical screening, prognosis, choice of therapy, evaluation of therapeutic benefit, i.e. for drug screening and drug development.

In one embodiment the disease is a condition involving pathological clinical complications of high levels of NETs or NETosis.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

Diagnostic or monitoring kits (or panels) are provided for performing methods of the invention. Such kits will suitably comprise one or more ligands for detection and/or quantification of the biomarker, optionally together with instructions for use of the kit.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Therefore, in a further aspect of the invention, the method may be performed as a near patient or point-of-care immunoassay method.

Methods of the invention permit integration of diagnostic procedures and therapeutic regimes. The method provides the means to indicate therapeutic response, failure to respond, unfavourable side-effect profile, degree of medication compliance and achievement of adequate serum drug levels. The method may be used to provide warning of adverse drug response. The method is useful in development of personalized therapies, as assessment of response can be used to fine-tune dosage, minimise the number of prescribed medications, reduce the delay in attaining effective therapy and avoid adverse drug reactions. Thus, by monitoring the subject, care can be tailored precisely to match the needs determined by the disorder and the pharmacological profile of the subject, and therefore be used to titrate the optimal dose, predict a positive therapeutic response and identify those subjects at high risk of severe side effects.

Biomarker monitoring methods, biosensors, point-of-care tests and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

References to “subject” or “patient” are used interchangeably herein. The subject may be a human or an animal subject. In one embodiment, the subject is a human. In one embodiment, the subject is a (non-human) animal. In a further embodiment, the animal is a companion animal (also referred to as a pet or domestic animal). Companion animals include, for example dogs, cats, rabbits, ferrets, horses, cows, or the like. In particular, the companion animal is a dog or cat, particularly a dog. The panels and methods described herein may be performed in vitro, or ex vivo.

Detecting and/or quantifying may be compared to a cut-off level. Cut-off values can be predetermined by analysing results from multiple patients and controls, and determining a suitable value for classifying a subject as with or without the disease. For example, for diseases where the level of biomarker is higher in patients suffering from the disease, then if the level detected is higher than the cut-off, the patient is indicated to suffer from the disease. Alternatively, for diseases where the level of biomarker is lower in patients suffering from the disease, then if the level detected is lower than the cut-off, the patient is indicated to suffer from the disease. The advantages of using simple cut-off values include the ease with which clinicians are able to understand the test and the elimination of any need for software or other aids in the interpretation of the test results. Cut-off levels can be determined using methods in the art.

Detecting and/or quantifying may also be compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject or a non-diseased subject, e.g. a subject without an infection. The control may also be a subject with the infection displaying no, or mild, symptoms, such as a subject infected with a respiratory virus displaying no, or mild, symptoms. Mild symptoms may include manageable symptoms which do not require hospital intervention and/or intensive medical treatment.

In one embodiment, a subject who tests positive by methods of the invention may be infected with a viral disease and additionally suffers, or goes on to suffer, further medical complications. In contrast, a control subject may also be infected with a viral disease but does not suffer, and does not go on to suffer, medical complications. Comparison with a control is well known in the field of diagnostics. The range of values found in the control group may be used as a normal or healthy or reference range against which the values found for test subjects can be compared. For example, if the reference range is <10 units, then a test value of 5 units would be considered normal, or not in need of treatment, but a value of 11 units would be considered abnormal and indicative of a need for treatment.

Therefore, in one embodiment, the method additionally comprises comparing the concentration of ETs (in particular, NETs), chromatin fragments and/or nucleosomes in the body fluid sample of the subject with one or more controls. The control may be a healthy subject. In one embodiment, the concentration of ETs, chromatin fragments and/or nucleosomes is elevated compared to the control.

It will be understood that it is not necessary to measure control levels for comparative purposes on every occasion. For example, for healthy/non-diseased controls, once the ‘normal range’ is established it can be used as a benchmark for all subsequent tests. A normal range can be established by obtaining samples from multiple control subjects without an infection and testing for the level of biomarker. Results (i.e. biomarker levels) for subjects suspected to have an infection can then be examined to see if they fall within, or outside of, the respective normal range. Use of a ‘normal range’ is standard practice for the detection of disease.

In one embodiment, the method additionally comprises determining at least one clinical parameter for the patient. This parameter can be used in the interpretation of results. Clinical parameters may include any relevant clinical information for example, without limitation, SOFA score, body temperature, gender, weight, Body Mass Index (BMI), smoking status and dietary habits. Therefore, in one embodiment, the clinical parameter is selected from the group consisting of: SOFA score, body temperature, age, sex and body mass index (BMI).

In one embodiment, the method of the invention is performed to identify a subject at high risk of developing a severe reaction to an infection and therefore in need of medical intervention. Such medical intervention may include one or more of the therapies as described herein.

According to another aspect of the invention, there is provided the use of a binding agent in the manufacture of a kit for use in a homogeneous immunoassay (HIA) method (e.g. as described herein) for detecting the concentration of ETs (such as NETs), chromatin fragments and/or nucleosomes in a body fluid sample.

### Infections

The methods of the current invention find particular use in managing infectious outbreaks. Infections can be caused by different pathogens and environmental factors. In one embodiment, the infection is a viral, bacterial, fungal or microbial infection. Bacterial infections may include mycobacterial, pneumococcal and influenzae infections, such as infections (e.g. pneumonia) caused by Streptococcus pneumoniae, Escherichia coli, Mycobacterium tuberculosis, Haemophilus influenzae and Staphylococcus aureus. In a further embodiment, the infection is a viral infection. Viral infections may include infections caused by respiratory syncytial virus (RSV), influenza type A, influenza type B and coronaviruses (e.g. COVID-19).

The infection can be defined by the tissue affected by the disease. For example, the disease may affect the heart, brain, kidneys, liver, pancreas, lungs and/or blood and the infection may be a bacterial, viral, fungal or microbial infection known to commonly affect such tissues or organs. In one embodiment, the infection is a respiratory tract infection. According to this embodiment, the infection affects the lungs, upper and/or lower respiratory tract.

Other tissues which may be affected by the disease include peripheral tissues such as limbs, hands and feet and the infection may be a bacterial infection (e.g. gangrene). In one embodiment, the infection and/or disease may affect multiple tissues or organs simultaneously. For example, the infection may be a bacterial infection of a limb, hand or foot and the disease may also affect the blood (e.g. sepsis). In one embodiment, the infection is sepsis. In another example, the disease may be cardiac or coronary failure and other tissues or organs affected by the disease may include the kidneys and renal system and/or the brain (e.g. stroke).

In a further embodiment, concentration of chromatin fragments is measured in a sample taken from a subject suffering from sepsis or septic shock, in particular to assess the prognosis of the disease. Further measurements on multiple samples taken at intervals from a subject suffering from sepsis or septic shock may be made to monitor the progress of the disease and/or to assess the efficacy of treatment.

In a yet further example, the disease may affect the lungs or the infection may be a respiratory tract infection and other tissues or organs affected may include the heart, coronary system and/or brain (e.g. heart failure, myocardial infarction and/or stroke). In one embodiment, the respiratory tract infection is selected from: influenza, pneumonia and severe acute respiratory syndrome (SARS). SARS is a respiratory infection caused by the SARS coronavirus (SARS-CoV) and other, related coronaviruses are known (e.g. COVID-19 (also known as SARS-COV-2 and previously as 2019-nCOV)). It is known to cause fever flu-like symptoms, cough and lethargy and can lead to pneumonia (e.g. direct viral pneumonia or secondary bacterial pneumonia).

NETs or NETosis related diseases include infectious diseases such as SIRS, sepsis, pneumonia, COVID and influenza as well as other diseases involving a pathological elevation in NETs production including, without limitation, pneumonia, SARS or ARDS of any cause, thrombotic or micro-thrombotic conditions, many inflammatory disease conditions and NETosis related complications of other diseases including amputation and thrombotic complications of diabetes and thrombotic complications of cancer. The method of the invention also has application in many other NETosis related diseases or conditions including for the ex vivo assessment of the viability of an organ or tissue removed from a donor patient for transplant to a recipient patient and for monitoring of a recipient patient for rejection of a transplanted organ or tissue.

Methods of Treatment of Subjects with Pathological Levels of NETs

According to a further aspect, there is provided a method of treating a disease (in particular, an infection) in a subject in need thereof, which comprises the step of administering a therapy (e.g. a therapeutic agent) to a subject identified as having a different concentration of ETs (such as NETs), chromatin fragments and/or nucleosomes in a sample obtained from said subject, when compared to the concentration of ETs, chromatin fragments and/or nucleosomes in a sample obtained from a control subject. The therapy may include one or more suitable treatments for the condition including without limitation, drugs (e.g. anti-inflammatory drugs, blood thinning or clotting inhibitor drugs, therapeutic anti-NETs antibody drugs, DNase drugs, NETosis inhibitor drugs, anti-bacterial drugs or anti-viral drugs), apheresis treatments, ventilator support, fluid support or others.

In one embodiment, the treatment is selected from one or more of: antibiotic treatments (e.g. penicillins, cephalosporins, tetracyclines, aminoglycosides, macrolides, clindamycin, sulphonamides, trimethoprim, metronidazole, tinidazole, quinolones and/or nitrofurantoin), anti-microbial treatments (e.g. ethambutol, isoniazid, pyrazinamide, rifampicin, aminoglycosides (amikacin, kanamycin), polypeptides (capreomycin, viomycin, enviomycin), fluoroquinolones (ciprofloxacin, levofloxacin, moxifloxacin), thioamides (ethionamide, prothionamide), cycloserine (closerin), terizidone, rifabutin, macrolides (clarithromycin), linezolid, thioacetazone, thioridazine, arginine, vitamin D and/or R207910), anti-viral COVID treatments (e.g. remdesivir), anti-viral influenza treatments (e.g. amantadine, umifenovir, moroxydine, rimantadine, umifenovir, zanamivir and neuraminidase inhibitors, cap-dependent endonuclease inhibitors, adamantanes, peramivir, zanamivir, oseltamivir phosphate and baloxavir marboxil) as well as anti-viral treatments for other viral diseases that may lead to a high level of NETosis and anti-fungal treatments (e.g. clotrimazole, econazole, miconazole, terbinafine, fluconazole, ketoconazole and amphotericin).

In one embodiment, the treatment is an anti-inflammatory drug. Many steroidal and non-steroidal anti-inflammatory drugs are known in the art. Some examples of steroidal anti-inflammatory drugs include without limitation, dexamethasone, hydrocortisone, cortisone, betamethasone, prednisone, prednisolone, triamcinolone and methylprednisolone. Some examples of non-steroidal anti-inflammatory drugs include without limitation, aspirin, celecoxib, diclofenac, diflunisal, etodolac, ibuprofen, indomethacin, CD24Fc (CD24 protein attached to the Fc region of immunoglobulin G) and EXO-CD24 (CD24-Exosomes).

In one embodiment, treatment is a DNase treatment to digest excess NETs or an inhibitor of NETosis, such as an anthracycline drug. In a further embodiment, the anthracycline drug is selected from: epirubicin, daunorubicin, doxorubicin and idarubicin.

In one embodiment the treatment is a therapeutic antibody drug directed to bind to NETs or to a component part of a NET including, without limitation, a therapeutic antibody directed to bind to a nucleosome, or to any component part of a nucleosome. Examples include therapeutic antibodies directed to bind to nucleosomes containing histone isoform H3.1, citrullinated histones, myeloperoxidase or neutrophil elastase.

In one embodiment the treatment is a nucleic acid scavenger that adsorbs and/or removes nucleic acids from the circulation or from the body, for example the DNA scavenger polyamidoamine.

A therapy involving the removal of NETs and NETs degradation products from the circulation by plasmapheresis that provides a NETosis treatment that avoids pharmaceutical toxic side effects has been reported (see WO2019/053243). Therefore, in one embodiment the treatment comprises plasmapheresis therapy.

In one embodiment, the infection is a respiratory infection, such as influenza or coronavirus, and the medical complication is pneumonia. Suitable treatments may include, without limitation, respiratory support using extracorporeal oxygenation, respiratory support using a medical ventilator designed to provide mechanical ventilation of air into and out of the lungs of a patient who is physically unable to breathe sufficiently unaided and/or provision of oxygen and/or antiviral, antibacterial or anti-inflammatory drugs.

### Haematological Cancer

Haematological cancers are the types of cancer affecting blood, bone marrow and lymph nodes. Common haematological cancers include, without limitation, leukaemia, lymphoma, myeloma and angiosarcoma depending on the type of cell affected. Haematological cancers are particularly common in companion animals, such as dogs or cats. Leukaemia is cancer of the blood cells which usually starts in the bone marrow and travels through the bloodstream. In leukaemia, the bone marrow produces mutated cells and spreads them into the blood, where they grow and crowd out healthy blood cells. Lymphoma diseases affect the cells in the lymphatic system. In lymphomas, immune cells called lymphocytes grow out of control and collect in lymph nodes, the spleen, in other lymph tissues or in neighbouring organs. Myeloma, also known as multiple myeloma, develops in the bone marrow and affects plasma cells, which produce antibodies that attack infections and diseases. Angiosarcoma (and hemangiosarcoma in canines) develop in the endothelia of the vasculature. Examples of blood cancers include Acute Lymphoblastic Leukaemia (ALL), Acute Myeloid Leukaemia (AML), Hodgkin Lymphoma (HL) and Non-Hodgkin Lymphoma (NHL). Common canine blood cancers include canine lymphoma and hemangiosarcoma.

Recent studies have shown there is a significant elevation in circulating cell free nucleosome levels found in patients with haematological cancers with median levels between 300-700 ng/ml in humans (WO2021110776) and up to 6000 ng/ml in canines (Dolan et al. BMC Veterinary Research (2021) 17:276 and Wilson-Robles et al. BMC Veterinary Research (2021) 17:231). These results indicate that an assay sensitivity in the range 100 ng/ml for nucleosomes is sufficient to usefully detect the presence of a haematological cancer in a patient or to monitor patient response to treatment. These levels are within the measurable range of HIA methods. The inventors propose using HIA techniques to provide low cost, rapid, near patient methods that can be used to make clinical decisions in real time.

As well as being useful in human subjects, the methods of the invention are useful in the diagnosis and treatment of subjects of other species, particularly canine subjects, with haematological cancer. This is in part because haematological cancers are common in dogs and in part because current methods involve scanning which require that the subject remain still in the scanner for the duration of the scan. Non-human subjects, such as dogs, will not remain still during a scan if conscious and must therefore be anaesthetised. The methods of the invention therefore provide a canine solution which is low cost, rapid, accurate and avoids the need to use a general anaesthetic.

Therefore, according to a further aspect of the invention, there is provided a method of detecting a haematological cancer disease in a subject, comprising:
(i) detecting the concentration of ETs, chromatin fragments and/or nucleosomes in a body fluid sample obtained from the subject using the method as defined herein;
(ii) using the concentration of ETs, chromatin fragments and/or nucleosomes as an indicator of the presence of a haematological cancer in the subject.

According to a further aspect of the invention, there is provided a method of detecting or monitoring the progress of a haematological cancer disease in a subject, comprising:
(i) detecting the concentration of ETs, chromatin fragments and/or nucleosomes in a body fluid sample obtained from the subject using the method as defined herein;
(ii) repeating step (i) on one or more occasions; and
(iii) using any changes in the concentration of ETs, chromatin fragments and/or nucleosomes to monitor the progression of the disease in the subject.

In one embodiment the subject with a haematological cancer is monitored to determine the efficacy of a treatment provided to the subject. It will be appreciated that, whilst methods of the invention are particularly useful for identifying and monitoring of patients with haematological cancers, application of the methods are not limited to haematological cancers but may be applied to any cancer.

Methods of Treatment of Subjects with Cancer

According to a further aspect, there is provided a method of treating a cancer, particularly a haematological cancer, in a subject in need thereof, which comprises the step of administering a therapy (e.g. a therapeutic agent) to a subject identified as having a different concentration of ETs, chromatin fragments and/or nucleosomes in a sample obtained from said subject, when compared to the concentration of ETs, chromatin fragments and/or nucleosomes in a sample obtained from a control subject. The therapy may include one or more suitable treatments for the condition including without limitation, drugs (e.g. cytotoxic drugs, chemotherapy drugs, immunotherapy drugs, epigenetic drugs and many more), radiotherapy treatments, blood transfusion treatments, blood dialysis treatments and apheresis treatments. If the haematological cancer involves metastases, then treatment may include surgery to remove secondary tumours.

### Kits

According to a further aspect of the invention, there is provided a kit for measuring the concentration of ETs (such as NETs), chromatin fragments and/or nucleosomes in a body fluid sample comprising: a reagent solution comprising one or more antibodies capable of specific binding to ETs, chromatin fragments and/or nucleosomes and a container suitable for use in a means to measure the degree of agglutination or precipitation caused upon antibody binding to determine the concentration of ETs, chromatin fragments and/or nucleosomes. Such kits may optionally include one or more buffer solutions.

Means to measure the degree of agglutination or precipitation caused upon antibody binding are well known in the art. For example, a spectrophotometer may be used to measure an increase in turbidity (i.e. a reduction in the intensity of light transmitted through the sample), which is due to the increasing particle size resulting from the agglutination reaction. Nephelometers may be used in nephelometric immunoassays to measure an increase in light scattering. Due to their easy one-step procedure and their short turn-around times, HIA methods are suitable for use in automated analysers, in particular clinical chemistry instruments.

According to a further aspect of the invention there is provided the use of the kit as defined herein for identifying a subject being susceptible to sepsis or septic shock, or for monitoring a subject with sepsis or septic shock. According to another aspect of the invention, there is provided the use of the kit as defined herein for identifying a subject with haematological cancer, or for monitoring a subject with haematological cancer.

### Additional Biomarkers

The concentration of NETs, chromatin fragments and/or nucleosomes may be detected or measured as one of a panel of measurements. The panel may comprise different epigenetic features of the nucleosome as described hereinbefore (e.g. a histone isoform and a PTM). Biomarkers useful in a panel test for the detection of severe respiratory infections that require medical intervention include, without limitation, cytokine moieties (particularly interleukins including, without limitation, IL-1, IL-6 and IL-8), C-reactive protein, myeloperoxidase, D-Dimer, factor VII-activating protease (FSAP), fibrinogen and fibrin/fibrinogen breakdown products. In one embodiment, the panel comprises C-reactive protein. In one embodiment, the panel comprises one or more cytokines, such as one or more interleukins.

It will be clear to those skilled in the art, that any combination of the biomarkers disclosed herein may be used in panels and algorithms (e.g. for monitoring the progress of a disease, such as an infection), and that further markers may be added to a panel including these markers.

According to an aspect of the invention there is provided the use of a panel test to detect or predict a complication to an infection in a patient, wherein the panel test comprises reagents to detect the concentration of ETs (such as NETs), chromatin fragments and/or nucleosomes in a homogeneous immunoassay method.

It will be understood that the embodiments described herein may be applied to all aspects of the invention, i.e. the embodiment described for the uses may equally apply to the claimed methods and so forth. The methods of the invention will now be illustrated by the following examples.

### Example 1

A nucleosome homogeneous immunoassay was performed using a Beckman Coulter AU400 analyser.

Agglutination was achieved using a combination of two different antibodies coated to latex particles. A first antibody was directed to bind to nucleosomes containing histone H3 isoform H3.1 (H3.1-nucleosomes) at an epitope that occurs near to amino acid 30 of H3 and was directly coated to latex particles (size 0.2 μm). A second antibody was directed to bind to a nucleosome conformational epitope only present in intact nucleosomes containing histones and DNA and was biotinylated and indirectly attached to Neutravidin coated latex particles (0.3 μm)

The assay procedure was as follows. A nucleosome test solution (25 μL) was added to phosphate buffer pH7.2 (150 μL) containing KCl, NaCl, detergent (Tween 20), polyethylene glycol 100, ethylenediaminetetraacetic acid (EDTA), bovine serum albumin and a preservative (sodium azide) for use. The mixture was incubated for 180 seconds at 37° C. A 0.2% suspension of latex particles in 50 mM Tris buffer (pH 8.0) containing sucrose, Tween 20, bovine serum albumin and sodium azide (30 μL) was added and incubated a further 36 seconds at 37° C. Agglutination of nucleosome bound latex particles was then measured kinetically as an increase in absorption of light at 700 nm over 270 seconds at 37° C.

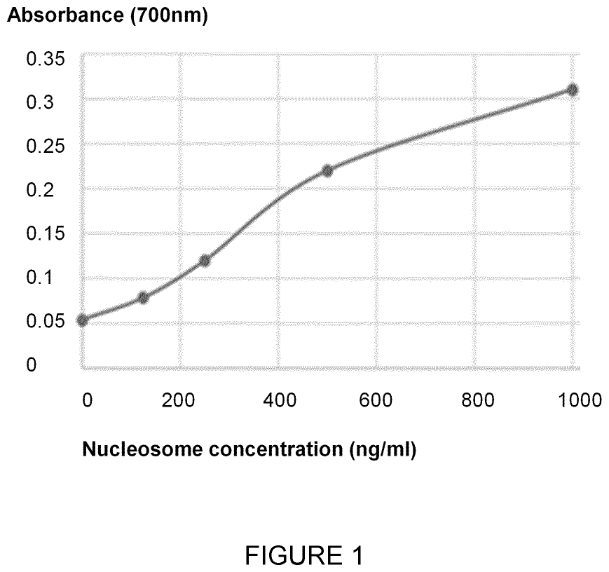

The results are shown in FIG. 1 and demonstrated that the measured agglutination increased with the concentration of nucleosomes present in the sample solution. The sensitivity of the assay is of the order of 100 ng/ml and this is adequate for the detection of nucleosomes in the context of diseases or conditions associated with NETosis including for example SIRS, ARDS, SARS, sepsis, pneumonia, COVID, influenza, thrombotic or micro-thrombotic conditions, inflammatory disease conditions and thrombotic conditions. The sensitivity is also adequate for applications in cancer, particularly in haematological cancers (myeloproliferative disorders), and also for use in many other areas including for assessment of ex vivo organ or tissue viability for transplant to a recipient patient and for monitoring of a recipient patient for rejection of a transplanted organ or tissue.

1. A method of detecting extracellular traps (ETs), chromatin fragments and/or nucleosomes in a body fluid sample, comprising:
analyzing the body fluid sample using a homogeneous immunoassay (HIA) method, and
using results obtained from said analyzing to determine a concentration of ETs, chromatin fragments and/or nucleosomes in the body fluid sample.

2. The method of claim 1, wherein the HIA method comprises:
(a) mixing the body fluid sample with a solution of one or more antibodies, wherein said antibodies are capable of specific binding to ETs, chromatin fragments and/or nucleosomes and cause agglutination or precipitation upon binding; and
(b) measuring a degree of agglutination or precipitation in the mixture;
wherein said measuring in step (b) is used to determine the concentration of ETs, chromatin fragments and/or nucleosomes in the body fluid sample.

3. The method of claim 2, wherein the one or more antibodies are present in a suspension of antibody coated particles.

4. The method of claim 2, wherein the one or more antibodies are conjugated to the surface of a latex bead or nanoparticle.

5. The method of claim 2, wherein the degree of agglutination or precipitation is measured by absorbance, transmittance, reflectance, light scatter, fluorescence, or scintillation of the mixture.

6. The method of claim 2, wherein the one or more antibodies are monoclonal or polyclonal antibodies.

7. (canceled)

8. The method of claim 1, wherein the HIA method is a turbidimetric immunoassay or nephelometric immunoassay.

9. The method of claim 1, wherein said body fluid sample is blood, serum or plasma.

10. The method of claim 9, wherein the blood, serum or plasma sample is obtained from a subject suffering, or suspected to be suffering, from sepsis, septic shock, or a haematological cancer.

11. (canceled)

12. The method of claim 1, wherein the ETs comprise neutrophil extracellular traps (NETs).

13. The method of claim 2, wherein the chromatin fragment and/or nucleosome comprises an epigenetic feature of a cell free nucleosome and the one or more antibodies are capable of specific binding to the epigenetic feature of the cell free nucleosome.

14. The method of claim 13, wherein the epigenetic feature is a histone isoform or a histone post translational modification (PTM).

15. The method as defined in claim 14, wherein the histone PTM is a histone PTM of a core nucleosome.

16. The method of claim 14, wherein the histone isoform is a histone H3 isoform or wherein the histone PTM is a histone PTM of a core nucleosome selected from citrullination and ribosylation.

17. The method of claim 2, wherein the one or more antibodies are capable of specific binding to a protein associated with a neutrophil extracellular trap (NET).

18. The method of claim 17, wherein the protein associated with a NET is selected from myeloperoxidase (MPO) and neutrophil elastase (NE).

19. A method of monitoring the progress of a disease in a subject, comprising:
(i) detecting a concentration of ETs, chromatin fragments and/or nucleosomes in a body fluid sample obtained from a subject using the method of claim 1;
(ii) repeating step (i) on one or more occasions; and
(iii) using any changes in the concentration of ETs, chromatin fragments and/or nucleosomes to monitor the progression of the disease in the subject.

20. A method of assigning a risk of an adverse outcome to a subject suffering from an infection, comprising:
(i) detecting a concentration of ETs, chromatin fragments and/or nucleosomes in a body fluid sample obtained from a subject using the method of claim 1; and
(ii) using the concentration of ETs, chromatin fragments and/or nucleosomes detected to assign the likelihood of an adverse outcome to said subject,
wherein a subject identified with a high likelihood of an adverse outcome is assigned for medical intervention.

21-26. (canceled)

27. A method of detecting a subject in need of medical treatment for sepsis or septic shock, comprising:
(i) detecting a concentration of ETs and/or chromatin fragments and/or nucleosomes in a body fluid sample obtained from the subject using the method of claim 1; and
(ii) using the concentration of ETs and/or chromatin fragments and/or nucleosomes as an indicator that the subject is in need of medical treatment for sepsis or septic shock.

28. A kit for measuring the concentration of ETs and/or chromatin fragments and/or nucleosomes in a body fluid sample comprising: a reagent solution comprising one or more antibodies capable of specific binding to ETs and/or chromatin fragments and/or nucleosomes and a container suitable for use in a means to measure the degree of agglutination or precipitation caused upon antibody binding to determine the concentration of ETs and/or chromatin fragments and/or nucleosomes, and optionally including one or more buffer solutions.

29-30. (canceled)
